(19) United States
(12) Patent Application Publication
(10) Pub. No.: US 2024/0245328 A1
(43) Pub. Date: Jul. 25, 2024

(54) NEAR-INFRARED SPECTROSCOPY BASED HANDHELD TISSUE OXYGENATION SCANNER

(71) Applicant: NEC Laboratories America, Inc., Princeton, NJ (US)

(72) Inventors: Sarper OZHARAR, Pennington, NJ (US); Thomas FERREIRA de LIMA, Princeton, NJ (US); Yue TIAN, Princeton, NJ (US); Wataru KOHNO, Princeton, NJ (US); Ting WANG, West Windsor, NJ (US)

(73) Assignee: NEC Laboratories America, Inc., Princeton, NJ (US)

(21) Appl. No.: 18/418,332

(22) Filed: Jan. 21, 2024

Related U.S. Application Data
(60) Provisional application No. 63/440,139, filed on Jan. 20, 2023.

(51) Int. Cl. A61B 5/1455; A61B 5/00

(52) U.S. Cl. CPC A61B 5/14552; A61B 5/742; A61B 2560/0431; A61B 2560/0468; A61B 2562/0219

(57) ABSTRACT

An improved near-infrared spectroscopy (NIRS) based handheld tissue oxygenation scanner employing a single transmitter-receiver pair, an integral inertial measurement sensor, an optional optical tracker, and wireless communications. Operational coordination among/between the elements are performed by a processor. The NIRS scanner device has a small foot print and permits the use of only a single transmitter/receiver pair to scan a large area of tissue manually, in real-time. Additional features and advantages of include battery power, hand held, may connect to a variety of computing devices via BlueTooth and/or WiFi, and may be modified to employ multiple transmitter/receivers to improve sensitivity and obtain absolute measurements.

### CROSS-REFERENCE TO RELATED APPLICATIONS

This application claims the benefit of U.S. Provisional Patent Application Ser. No. 63/440,139 filed Jan. 20, 2023, the entire contents of which is incorporated by reference as if set forth at length herein.

### FIELD OF THE INVENTION

This application relates generally to near-infrared spectroscopy More particularly, it pertains to near-infrared spectroscopy based handheld tissue oxygenation scanner.

### BACKGROUND OF THE INVENTION

As is known in the art, near-infrared spectroscopy (NIRS) is a technology to measure the changes in the oxyhemoglobin and deoxyhemoglobin in both arterial and venous blood, and the total hemoglobin in tissue.

Methods and devices employing NIRS technology send near infrared and infrared (red) light into the tissue and detect any backscattered light. By comparing changes and ratios of intensities of backscattered light, operational methods and devices employing the same may determine the oxy- and deoxy-hemoglobin in the tissue that is illuminated.

Such methods and devices employing NIRS technology are known to be quite useful in diagnostic environments. Accordingly, improvements in such methods and devices would be a welcome addition to the art.

### SUMMARY OF THE INVENTION

An advance in the art is made according to aspects of the present disclosure directed to an improved near-infrared spectroscopy based handheld tissue oxygenation scanner.

In sharp contrast to the prior art, our inventive handheld tissue oxygenation scanner is a small device—like an optical mouse—employing a single transmitter-receiver pair, an integral inertial measurement sensor, a tracker, and wireless communications. Operational coordination among/between the elements are performed by a processor.

Our inventive integration of motion tracking into a NIRS device having a small foot print permits the use of only a single transmitter/receiver pair to scan a large area of tissue manually, in real-time. Additional features and advantages of our inventive include battery powered, hand held, may connect to a variety of computing devices via BlueTooth, and may be modified to employ multiple transmitter/receivers to improve sensitivity and obtain absolute measurements.

### BRIEF DESCRIPTION OF THE DRAWING

A more complete understanding of the present disclosure may be realized by reference to the accompanying drawing in which:

FIG. 1 is a schematic diagram showing an illustrative side view of a tissue oxygenation scanner according to aspects of the present disclosure;

FIG. 2 is a schematic diagram showing an illustrative operation of a tissue oxygenation scanner according to aspects of the present disclosure;

FIG. 3 is a schematic flow diagram showing an illustrative operational flow of a tissue oxygenation scanner according to aspects of the present disclosure;

FIG. 4 is a schematic diagram showing illustrative features of a tissue oxygenation scanner according to aspects of the present disclosure;

FIG. 5 is a schematic diagram showing an illustrative “mouse” configuration of a tissue oxygenation scanner according to aspects of the present disclosure; and

FIG. 6 is a schematic diagram showing an illustrative computer architecture of a tissue oxygenation scanner according to aspects of the present disclosure.

The illustrative embodiments are described more fully by the Figures and detailed description.

The inventions may however, be embodied in various forms and are not limited to specific embodiments described in the Figures and detailed description.

### DETAILED DESCRIPTION OF THE INVENTION

The following merely illustrates the principles of this disclosure. It will thus be appreciated that those skilled in the art will be able to devise various arrangements which, although not explicitly described or shown herein, embody the principles of the disclosure and are included within its spirit and scope.

Furthermore, all examples and conditional language recited herein are intended to be only for pedagogical purposes to aid the reader in understanding the principles of the disclosure and the concepts contributed by the inventor(s) to furthering the art and are to be construed as being without limitation to such specifically recited examples and conditions.

Moreover, all statements herein reciting principles, aspects, and embodiments of the disclosure, as well as specific examples thereof, are intended to encompass both structural and functional equivalents thereof. Additionally, it is intended that such equivalents include both currently known equivalents as well as equivalents developed in the future, i.e., any elements developed that perform the same function, regardless of structure.

Thus, for example, it will be appreciated by those skilled in the art that any block diagrams herein represent conceptual views of illustrative circuitry embodying the principles of the disclosure.

Thus, for example, it will be appreciated by those skilled in the art that any block diagrams herein represent conceptual views of illustrative circuitry embodying the principles of the disclosure. Similarly, it will be appreciated that any flow charts, flow diagrams, state transition diagrams, pseudo code, and the like represent various processes which may be substantially represented in computer readable medium and so executed by a computer or processor, whether or not such computer or processor is explicitly shown.

The functions of the various elements shown in the Figures, including any functional blocks labeled as “processors”, may be provided through the use of dedicated hardware as well as hardware capable of executing software in association with appropriate software. When provided by a processor, the functions may be provided by a single dedicated processor, by a single shared processor, or by a plurality of individual processors, some of which may be shared. Moreover, explicit use of the term “processor” or “controller” should not be construed to refer exclusively to hardware capable of executing software, and may implicitly include, without limitation, digital signal processor (DSP) hardware, network processor, application specific integrated circuit (ASIC), field programmable gate array (FPGA), read-only memory (ROM) for storing software, random access memory (RAM), and non-volatile storage. Other hardware, conventional and/or custom, may also be included.

Software modules, or simply modules which are implied to be software, may be represented herein as any combination of flowchart elements or other elements indicating performance of process steps and/or textual description. Such modules may be executed by hardware that is expressly or implicitly shown.

Unless otherwise explicitly specified herein, the FIGs comprising the drawing are not drawn to scale.

Generally, and as those skilled in the art will understand and appreciate, near-infrared spectroscopy (NIRS) is a powerful analytical technique that uses the near-infrared region of the electromagnetic spectrum (780 nm to 2500 nm) to identify and quantify the chemical composition of a sample substance. It works by measuring how much light at different wavelengths is absorbed by the sample.

NIRS has many advantages over other analytical techniques, such as:
Non-destructive: It does not harm or alter the sample.
Fast: Measurements can be made quickly and easily.
Versatile: It can be used to analyze a wide variety of materials, including liquids, solids, and gases.
Portable: NIR spectrometers can be small and portable, making them suitable for field use.

NIRS has a wide range of applications in various fields, including:
Food and agriculture: Analyzing the quality and composition of food products, monitoring crop growth, and detecting contaminants.
Pharmaceuticals: Identifying and quantifying the active ingredients in drugs, monitoring drug production processes, and ensuring the quality of pharmaceutical products.
Chemical industry: Analyzing the composition of chemicals, monitoring chemical reactions, and optimizing production processes.
Environmental monitoring: Detecting pollutants in air and water, monitoring soil quality, and analyzing the composition of plants.
Medical diagnostics: Measuring blood oxygen levels in the brain, monitoring brain activity, and diagnosing diseases such as cancer.

Overall, NIRS is a powerful and versatile analytical technique that has a wide range of applications. It is a valuable tool for scientists, engineers, and technicians in many different fields.

As noted previously, an aspect of the present disclosure is directed to an improved near-infrared spectroscopy based handheld tissue oxygenation scanner that is a small, handheld, portable device—like an optical mouse—employing a transmitter-receiver pair, an integral inertial measurement sensor, a position tracker, and wireless communications. Operational coordination among/between the elements are performed by a processor.

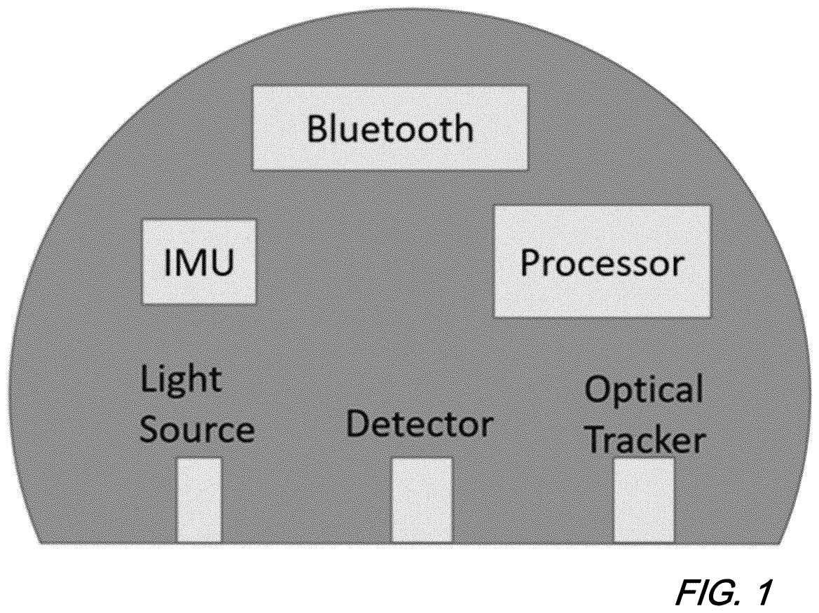

FIG. 1 is a schematic diagram showing an illustrative side view of a tissue oxygenation scanner according to aspects of the present disclosure.

With reference to that figure, one may readily recognize some particularly distinguishing aspects of the improved near-infrared spectroscopy based handheld tissue oxygenation scanner according to the present disclosure. As depicted, the scanner is small, handheld, portable device-like an optical mouse-and may include only a single transmitter-receiver pair thereby reducing cost.

An optical tracker, an integral inertial measurement sensor, a position tracker, and wireless communications (i.e., Bluetooth) are all components of our scanner. Operational coordination among/between the elements are performed by a processor.

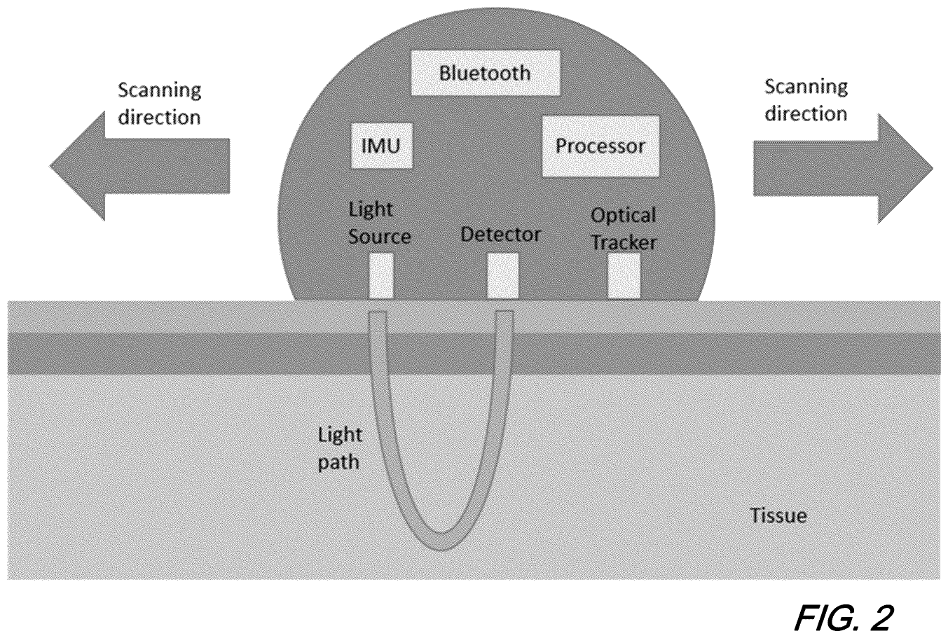

FIG. 2 is a schematic diagram showing an illustrative operation of a tissue oxygenation scanner according to aspects of the present disclosure.

Operationally, the light source emits near-infrared light which is directed along a light path into sample tissue such as human skin or other tissue(s). As noted previously, the light sources emit light at specific wavelengths and may comprise, for example, light-emitting diodes (LEDs).

When the emitted light traverses the tissue, hemoglobin, the protein in red blood cells that carries oxygen, absorbs the light differently depending on whether it is carrying oxygen, i.e., oxygenated, (oxyhemoglobin), or not (deoxyhemoglobin). Oxyhemoglobin absorbs more infrared light than deoxyhemoglobin.

The scanner also includes a light detector that detects and allows measurement of the amount of light that passes through the tissue and exits that tissue.

By comparing the amount of light absorbed at different wavelengths, the scanner can calculate the relative amounts of oxyhemoglobin and deoxyhemoglobin in the tissue. This information may then be used to calculate a value called tissue oxygen saturation (StO2), which is a measure of the percentage of hemoglobin molecules that are carrying oxygen.

When the scanner device starts measurement, oxy-and deoxy-hemoglobin values within illuminated regions of tissue are measured, and as the scanner device is moved around along the tissue, this motion is tracked and added to the measured oxy-deoxy-values.

As we noted, NIRS has several advantages over other methods of measuring oxygenation, such as pulse oximetry:
Non-invasive: NIRS does not require breaking the skin, making it a more comfortable and convenient method for patients.
Can measure deeper tissues: NIRS can measure oxygenation in deeper tissues, such as the brain and muscles, which is not possible with pulse oximetry.
Continuous monitoring: NIRS can provide continuous monitoring of oxygenation, which is useful for monitoring patients in critical care settings.

Also included and shown in the figure is an optional optical tracker, which may itself be an single integrated circuit that provides a light source, a lens that focuses the light beam onto the tissue surface where the scanner is moving, and tracker sensor that may be—for example—a CMOS (Complementary Metal-Oxide-Semiconductor) sensor that captures the reflected light from the tissue surface and determines movement of the NIRS across the surface of the tissue. Generally, such a optical tracking sensor is made up of tiny photosensitive pixels that convert light into electrical signals. The electrical signals from the sensor are processed to determine the movement of the sensor. The IPU calculates the relative displacement of the light pattern on the sensor, which corresponds to the movement of the mouse on the surface.

Further shown in the figure is an inertial measurement unit (IMU), which is electronic circuitry that measures and reports the motion of an object. As previously noted, the IMU may be employed with or without the optical tracker. An IMU may report various motion, including acceleration, angular velocity, and sometimes orientation. It achieves this by combining the data from multiple sensors, typically:
Accelerometers: These measure the linear acceleration of the object in three axes (forward/backward, left/right, up/down);
Gyroscopes: These measure the angular velocity of the object in three axes (roll, pitch, yaw); and
Magnetometers (optional): These measure the strength and direction of the Earth's magnetic field, which can be used to determine the heading of the object.

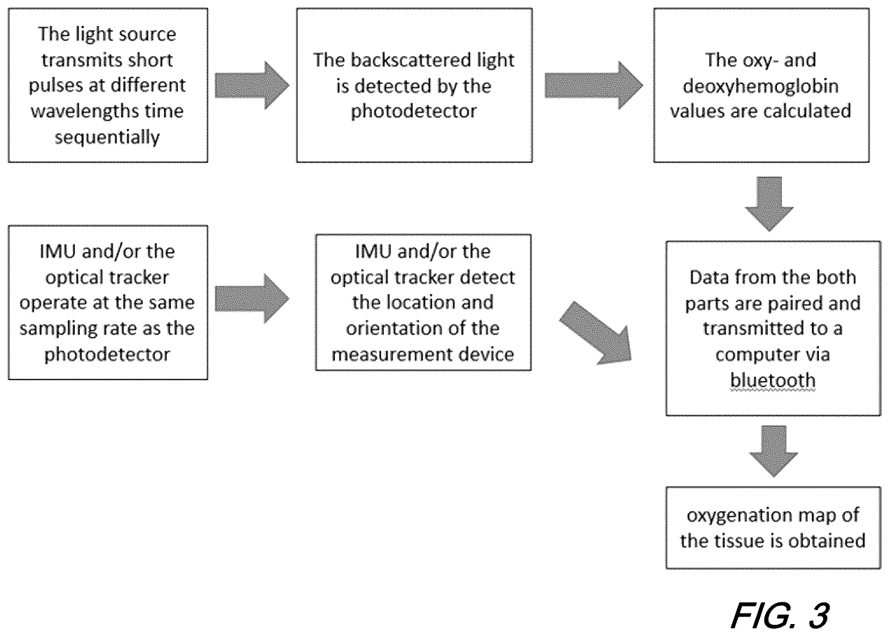

FIG. 3 is a schematic flow diagram showing an illustrative operational flow of a tissue oxygenation scanner according to aspects of the present disclosure. To discuss this figure, we first review the operational circuitry elements namely, NIRS transmitter receiver circuitry pair that obtain the oxygenation of the tissue under investigation; motion tracking circuitry system employing an IMU and/or an optical tracking similar to an optical mouse; processor/processing circuitry, that combines the NIRS data with the location data, hence creating a 2D map of the oxygenation of the measured tissue; and wireless communication circuitry, that transmits the measurement results wirelessly (via Wi-Fi or Bluetooth) to a nearby computer.

When the device starts measurement, part 1 calculates the oxy-and deoxy-hemoglobin values at the illuminated area. As the device is moved around along the tissue, this motion is tracked and added to the measured oxy-deoxy-values.

As illustratively shown in FIG. 3, the light source transmits (short pulses) of light at different wavelengths time sequentially. The backscattered light is detected by the photodetector and the oxy- and deoxyhemoglobin values are determined.

Simultaneously, an IMU and/or optical tracker circuitry operate at the same sampling rate as the photodetector. The IMU and/or optical tracker detects the location and orientation of the NIRS scanner device.

Both oxy-deoxy-data and IMU and/or tracker data are paired and transmitted to a computer via wireless mechanisms such as BlueTooth or WiFi.

Processing at the remote computer may produce an oxygenation map of the tissue. Alternatively, and depending on the particular implementation, the map may be produced in the NIRS scanner and then transmitted as a map via wireless.

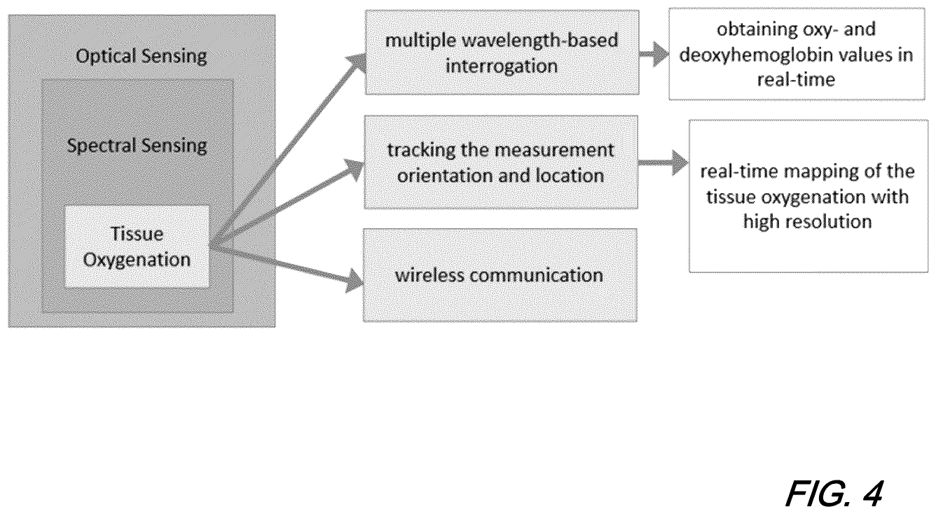

FIG. 4 is a schematic diagram showing illustrative features of a tissue oxygenation scanner according to aspects of the present disclosure.

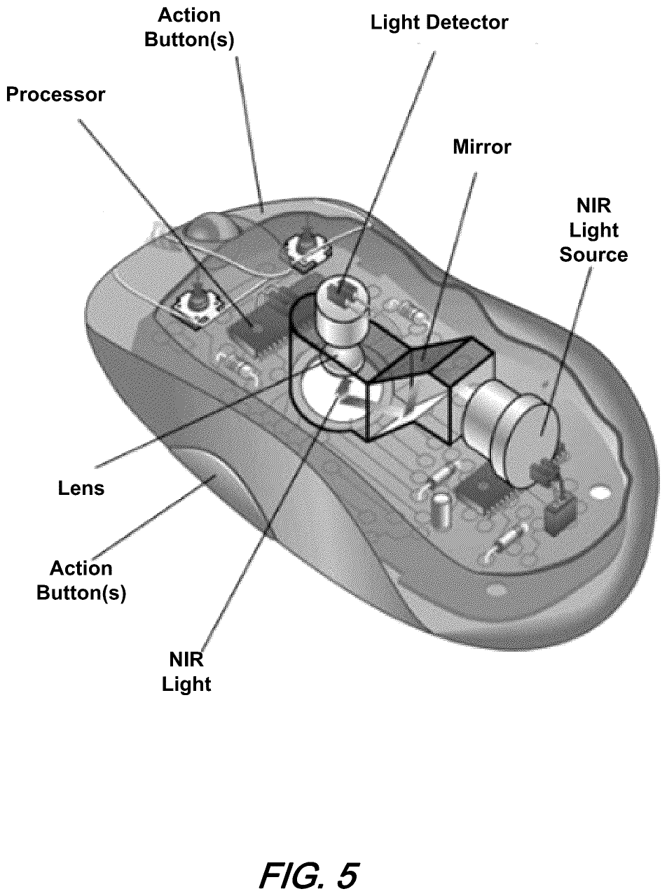

FIG. 5 is a schematic diagram showing an illustrative “mouse” configuration of a tissue oxygenation scanner according to aspects of the present disclosure. As illustratively shown in that figure, the NIRS scanner may have a familiar configuration and feel as a computer mouse. As may be appreciated, such device may be battery powered resulting in completely portable scanner.

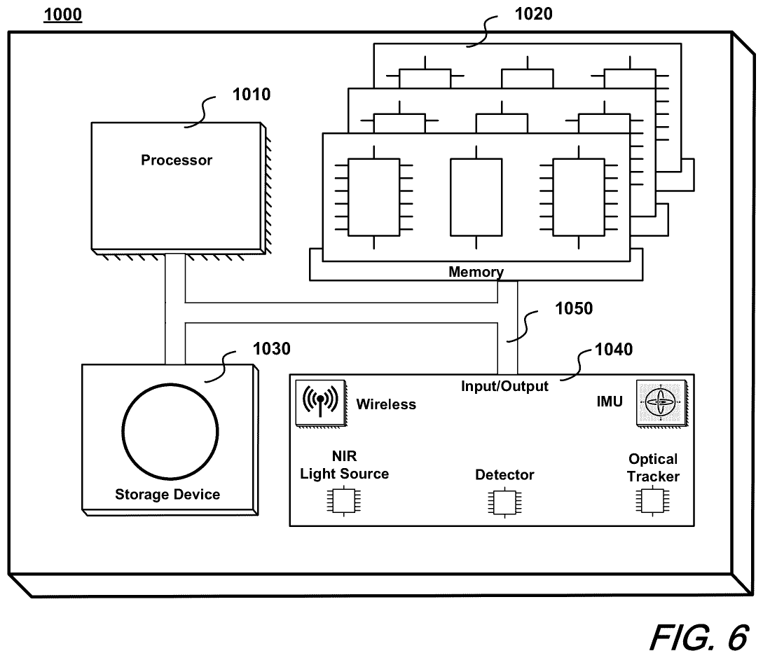

FIG. 6 is a schematic diagram showing an illustrative computer architecture of a tissue oxygenation scanner according to aspects of the present disclosure. As may be immediately appreciated, such a system may be integrated into a variety of sensor probe configurations, and may be implemented via discrete circuitry elements or one or more integrated circuitry components. The computer system may comprise, for example a computer running any of a number of operating/control programs. The above-described methods of the present disclosure may be implemented on the computer system 1000 as stored program control instructions.

Computer system 1000 includes processor 1010, memory 1020, storage device 1030, and input/output structure 1040. One or more input/output devices may include a display 1045. One or more busses 1050 typically interconnect the components, 1010, 1020, 1030, and 1040. Processor 1010 maybe a single or multi core. Additionally, the system may include accelerators etc further comprising the system on a chip.

Processor 1010 executes instructions in which embodiments of the present disclosure may comprise steps described in one or more of the Drawing figures. Such instructions may be stored in memory 1020 or storage device 1030. Data and/or information may be received and output using one or more input/output devices.

Memory 1020 may store data and may be a computer-readable medium, such as volatile or non-volatile memory. Storage device 1030 may provide storage for system 1000 including for example, the previously described methods. In various aspects, storage device 1030 maybe a flash memory device, a disk drive, an optical disk device, or a tape device employing magnetic, optical, or other recording technologies.

Input/output structures 1040 may provide input/output operations for system 1000 and include any number of structures previously described including wireless circuitry, light source(s), detector circuitry, optical tracker circuitry, and IMU circuitry.

At this point, while we have presented this disclosure using some specific examples, those skilled in the art will recognize that our teachings are not so limited. Accordingly, this disclosure should only be limited by the scope of the claims attached hereto.

1. A near-infrared tissue oxygenation scanner comprising:
a mechanical housing containing
a near-infrared light source that emits near-infrared light;
an optical detector that detects the emitted near-infrared light after the emitted near-infrared light is absorbed and/or scattered by tissues (NIRS data);
motion tracking circuitry configured to detect motion of the scanner during operation and collect motion tracking data; and
a processor that determines oxy-and deoxyhemoglobin values from the NIRS data and combines those values with collected motion tracking data for wireless transmission via wireless communication circuitry.

2. The scanner of claim 1 wherein the motion tracking circuitry includes one or more of an inertial measurement circuitry included in an IMU and an optical tracker.

3. The scanner of claim 2 wherein the processor generates a 2D map of the oxygenation of the tissue.

4. The scanner of claim 2 wherein the wireless communication circuitry includes one or more of Bluetooth and WiFi communication circuitry.

5. The scanner of claim 1 wherein the near-infrared light source generates multiple wavelengths simultaneously.
